(19) United States
(12) Patent Application Publication
(10) Pub. No.: US 2014/0100511 A1
(43) Pub. Date: Apr. 10, 2014

(54) NOVEL PRESSURE EQUALIZATION TUBE

(71) Applicant: GYRUS ACMI, INC.,D.B.A. OLYMPUS SURGICAL TECHNO, Southborough, MA (US)

(72) Inventor: Phillip A. Ryan, Memphis, TN (US)

(73) Assignee: Gyrus ACMI, Inc., d.b.a. Olympus Surgical Technologies America, Southborough, MA (US)

(21) Appl. No.: 13/647,373

(22) Filed: Oct. 8, 2012

(51) Int. Cl. A61F 11/00; A61M 1/00

(52) U.S. Cl. 604/8

(57) ABSTRACT

A pressure equalization tube assembly for insertion through a membrane of an ear is provided. The pressure equalization tube assembly includes a tube having an interior wall surface extending between a first end and a second end. The interior wall surface defines a lumen extending between the first and second ends. A first part extends from the first end of the tube and a second part extends from the second end of the tube. The first and second parts may be wire portions, coil portions, and/or superelastic flanges that are moveable between a collapsed state and an expanded state.

### FIELD

The present disclosure relates to a pressure equalization tube, and more particularly, to a pressure equalization tube that can be inserted into a tympanic membrane to vent differential pressure build-up across the tympanic membrane.

### BACKGROUND

The statements in this section merely provide background information related to the present disclosure and may or may not constitute prior art.

A pressure equalization or vent tube is used in a human ear to ventilate the ear and prevent fluid build-up. The pressure equalization tube is inserted through the tympanic membrane, or eardrum, of the patient, who is typically a small child. The pressure equalization tube remains in the ear for a prolonged period of time, such as, for example, six months to two years. The pressure equalization tube may then fall out or be removed.

A common design for a pressure equalization tube has a grommet shape. However, such grommet-shaped pressure equalization tubes have large flanges on each end that require a fairly large incision in the tympanic membrane in order to be inserted through the tympanic membrane, which may be undesirable. Accordingly, there exists a need for a pressure equalization tube that may be inserted without making a large incision in the tympanic membrane.

### SUMMARY

The present disclosure provides a novel pressure equalization tube that includes an inner tube attached to outer portions that may be collapsible to insert the tube assembly through a small incision of the tympanic membrane, if desired. The outer portions may be flanges that are formed of a wire coils.

In one aspect, which may be combined with or separate from the other aspects described herein, the present disclosure provides a pressure equalization tube assembly for insertion through a membrane of an ear. The pressure equalization tube assembly may include a tube, a first wire portion, and a second wire portion. The tube may have an interior wall surface extending between a first end and a second end, wherein the interior wall surface defines a lumen extending between the first and second ends. The first wire portion may extend from the first end of the tube, and the second wire portion may extend from the second end of the tube.

In another aspect, which may be combined with or separate from the other aspects described herein, the present disclosure provides a pressure equalization tube assembly for insertion through a membrane of an ear. The pressure equalization tube assembly may include a tube, a first coil portion, and a second coil portion. The tube may have an interior wall surface extending between a first end and a second end, wherein the interior wall surface defines a lumen extending between the first and second ends. The first coil portion may extend from the first end of the tube, and the second coil portion may extend from the second end of the tube.

In yet another aspect, which may be combined with or separate from the other aspects described herein, the present disclosure provides a pressure equalization tube assembly for insertion through a tympanic membrane of an ear. The pressure equalization tube assembly may include a tube, a first flange, and a second flange. The tube may have an interior wall surface extending between a first end and a second end, wherein the interior wall surface defines a lumen extending between the first and second ends. The first flange may extend from the first end of the tube, and the first flange may be superelastic and movable between a collapsed state and an expanded state. The second flange may extend from the second end of the tube, and the second flange may be superelastic and movable between a collapsed state and an expanded state.

Accordingly, pursuant to one aspect of the invention, there is contemplated a pressure equalization tube assembly for insertion through a membrane of an ear, the pressure equalization tube assembly comprising one or more of the following: a tube having an interior wall surface extending between a first end and a second end, the interior wall surface defining a lumen extending between the first and second ends; a first wire portion extending from the first end of the tube; and a second wire portion extending the second end of the tube.

Accordingly, pursuant to another aspect of the invention, there is contemplated a pressure equalization tube assembly for insertion through a membrane of an ear, the pressure equalization tube assembly comprising one or more of the following: a tube having an interior wall surface extending between a first end and a second end, the interior wall surface defining a lumen extending between the first and second ends; a first coil portion extending from the first end of the tube; and a second coil portion extending the second end of the tube.

Accordingly, pursuant to yet another aspect of the invention, there is contemplated a pressure equalization tube assembly for insertion through a tympanic membrane of an ear, the pressure equalization tube assembly comprising one or more of the following: a tube having an interior wall surface extending between a first end and a second end, the interior wall surface defining a lumen extending between the first and second ends; a first flange extending from the first end of the tube, the first flange being superelastic and being movable between a collapsed state and an expanded state; and a second flange extending from the second end of the tube, the second flange being superelastic and being movable between a collapsed state and an expanded state.

The invention may be further characterized by one or any combination of the features described herein, such as: the first and second wire portions or flanges are coils; the first and second wire portions, coil portions, or flanges comprise a superelastic material; the first and second wire portions, coil portions, or flanges are formed of Nitinol; the first and second wire portions, coil portions, or flanges extend through the tube; the first and second wire portions, coil portions, or flanges are integrally formed as one piece; the first wire portion, coil portion, or flange extends substantially within a first plane and the second wire portion, coil portion, or flange extends substantially within a second plane when the pressure equalization tube assembly is placed through the membrane in a working configuration; the tube has a circular cylindrical shape; the tube is formed of a polymeric material; the first flange defines a first expanded outer diameter in the expanded state, the second flange defines a second expanded outer diameter in the expanded state, the tube defines a tube outer diameter, the first flange defines a first collapsed outer diameter in the collapsed state, and the second flange defines a second collapsed outer diameter in the collapsed state, the first and second expanded outer diameters each being greater than the tube outer diameter, and the first and second expanded outer diameters each being greater than each of the first and second collapsed outer diameters; the first and second collapsed outer diameters are each no greater than twice as large as the tube outer diameter; and the first and second flanges are formed unitarily as one piece from a Nitinol wire.

Further aspects, advantages and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a schematic perspective view of a pressure equalization tube assembly in an expanded configuration, in accordance with the principles of the present disclosure;

FIG. 2 is a side cross-sectional view of a delivery device disposed around the pressure equalization tube assembly of FIG. 1 in a collapsed configuration, according to the principles of the present disclosure; and

FIG. 3 is a cross-sectional view of the pressure equalization tube assembly of FIGS. 1-2 in an expanded configuration and inserted within an ear, in accordance with the principles of the present disclosure.

### DETAILED DESCRIPTION

The following description is merely exemplary in nature and is not intended to limit the present disclosure, application, or uses.

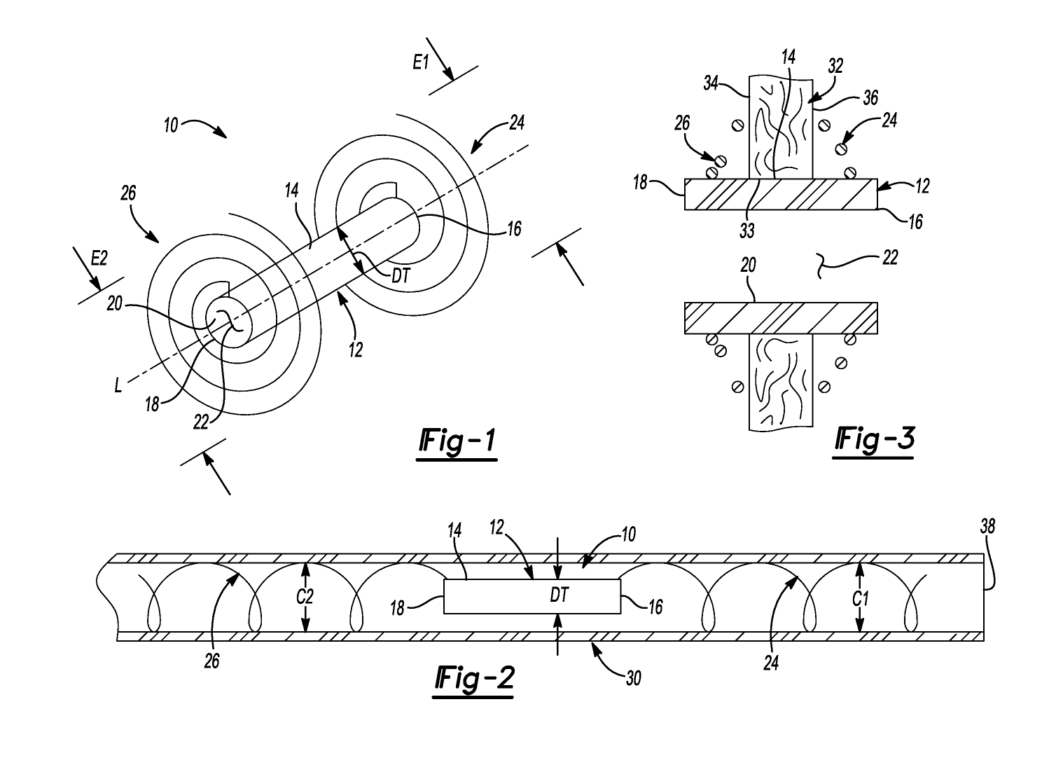

With reference to the figures, wherein like numerals indicate like components, and specifically with reference to FIG. 1, an example of a pressure equalization tube assembly in accordance with the principles of the present disclosure is illustrated and generally designated at 10. The pressure equalization tube assembly 10 is intended to be inserted through a tympanic membrane of an ear to ventilate and/or drain the middle ear.

The pressure equalization tube assembly 10 has a tube 12 that includes a main body portion 14 extending between first and second ends 16, 18 along a longitudinal axis L. The tube 12 has a generally hollow circular cylindrical shape. Thus, the tube 12 has an inner wall 20 that defines a lumen 22 through the main body portion 14 and the first and second ends 16, 18. The first and second ends 16, 18 are open ends such that the lumen 22 is open to environment adjacent to the ends 16, 18.

The tube 12 may be formed of a polymeric material, such as polyethylene (PE), polytetrafluoroethylene (PTFE), such as Teflon, Silicone, or other fluoroplastics, though other materials may also or alternatively be used. Coating and treatment may also be applied to the tube 12 to minimize biofilm adhesion.

A first coil portion 24 extends from the first end 16 of the tube 12, and a second coil portion 26 extends from the second end 18 of the tube 12. Each of the coil portions 24, 26 is a spiral coil that extends from the ends 16, 18 of the tube 12. The coil portions 24, 26 may be formed together unitarily as one-piece of wire that extends through the tube 12 and protrudes through both ends 16, 18 of the tube 12. The one piece of wire that forms the coil portions 24, 26 may be formed through the tube 12 to secure the coil portions 24, 26 to the tube 12; or, the one piece of wire may be secured to the tube 12 in another suitable manner. Alternatively, the coil portions 24, 26 may be formed from two or more separate pieces of wire or other material, which may also be formed through the tube 12 or otherwise attached to the tube 12.

The first and second coil portions 24, 26 comprise a superelastic material, or a shape memory material, such as Nitinol. The superelastic material may be configured to return to its original position after being deflected. Accordingly, the first and second coil portions 24, 26 may be preformed into helical, coil shapes extending from each end 16, 18 of the tube 12. The first and second coil portions 24, 26 may be compressible into a collapsed state, and the first and second coil portions 24, 26 may be configured to automatically self-expand into an expanded state to return to the preformed helical, coil shapes. The first and second coil portions 24, 26 may be elastically deformed, or squashed down, in a delivery device, and the first and second coil portions 24, 26 may be configured to return to their original preformed shapes after being deployed from the delivery device. For example, the first and second coil portions 24, 26 could be pulled or stretched into a straight or wavy wire in the delivery device, and the first and second coil portions 24, 26 could be configured to thereafter return to the preformed coil shapes when no longer loaded or constrained.

Thus, with reference to FIG. 2, the coil portions 24, 26 may be collapsed to fit into a narrow delivery tube 30. In the illustrated embodiment, the narrow delivery tube 30 has a cylindrical shape. The first and second coil portions 24, 26, which form first and second flanges of the pressure equalization tube assembly 10, are configured in a collapsed state that to allow the pressure equalization tube assembly 10 to fit into the delivery tube 30.

Referring to FIGS. 2 and 3, in one example, to insert the pressure equalization tube assembly 10 into the tympanic membrane 32, a small incision 33 is made in the tympanic membrane 32, for example, with a trocar (not shown) through an outer side 34 and an inner side 36 of the tympanic membrane 32. The trocar may be integrally formed with the delivery tube 30, or the trocar may be a separate instrument. The delivery tube is advanced through the incision 33, which extends between the outer side 34 and the inner side 36 of the tympanic membrane 32. Once the delivery tube 30 is inserted through both sides 34, 36 of the tympanic membrane 32 into the middle ear, the first coil portion 24 is deployed from the distal end 38 of the delivery tube 30. The first coil portion 24 may then self-expand into its expanded state, as shown in FIGS. 1 and 3. The delivery tube 30 is then retracted through the incision 33, and the tube 12 and second coil portion 26 are deployed from the distal end 38 of the delivery tube 30. Upon being deployed from the delivery tube 30, the second coil portion 26 also self-expands from the collapsed state (as shown in FIG. 2) to the expanded state (as shown in FIGS. 1 and 3). The delivery tube 30 is then withdrawn from the ear. The result is that the pressure equalization tube assembly 10 is deployed through the tympanic membrane 32 without having to make a large incision in the tympanic membrane to fit the expanded configuration of the flanges 24, 26 of the assembly 10 through the tympanic membrane 32. Instead, the flanges 24 are compressed into their collapsed states to fit through a small incision 33 of the tympanic membrane 32.

In the expanded state, the first and second flanges 24, 26 (also referred to as coil portions 24, 26 herein) have outer expanded diameters E1, E2 that are each larger than the outer diameter DT of the tube 12. The expanded diameters E1, E2 of the first and second flanges 24, 26 are also each greater than each of the collapsed diameters C1, C2 of the first and second flanges 24, 26. Each collapsed diameter C1, C2 may be greater than the tube outer diameter DT. In some variations, each collapsed diameter C1, C2 is no greater than twice as large as the tube outer diameter DT. In other words, C1 is less than or equal to 2×DT; and C2 is less than or equal to 2×DT, in some embodiments. The outer diameter DT of the tube 12 may be about one millimeter, by way of example. Thus, the collapsed diameters C1, C2 could be less than or equal to about two millimeters, in some embodiments. The expanded diameters E1, E2 could be larger than two millimeters, in this example, such as between 2-5 millimeters or larger.

In some variations, the geometry of the coil portions 24, 26 could be modified to adjust the strength of contact between the coil portions 24, 26 and the sides 34, 36 of the tympanic membrane 32. For example, the angle that the coil portions 24, 26 extend from the tube 12 could be increased or decreased. In the illustrated embodiment of FIG. 1, the first coil portion 24 extending substantially within a first plane; likewise, the second coil portion 26 extends substantially within a second plane. As such, the coil portions 24 form flanges that are substantially perpendicular to a longitudinal axis L of the tube 12.

In another variation, the tube 12 could be formed of a metal, such as Nitinol. In one variation, the tube 12 could be unitarily formed with the coil portions 24, 26 such that the tube 12 and coil portions 24, 26 are formed of a single piece of Nitinol material. For example, the tube 12 could be a Nitinol (or other metal) tube that is spiral cut on both ends 16, 18 to form the coil portions 24, 26.

The description of the invention is merely exemplary in nature and variations that do not depart from the gist of the invention are intended to be within the scope of the invention. Such variations are not to be regarded as a departure from the spirit and scope of the invention.

1-15. (canceled)

16. A method of ventilating a membrane of an ear, the method comprising inserting a pressure equalization tube assembly through a membrane of an ear, the pressure equalization tube assembly comprising a tube having an interior wall surface extending between a first end and a second end, the interior wall surface defining a lumen extending between the first and second ends, a first wire portion extending from the first end of the tube, and a second wire portion extending from the second end of the tube.

17. The method of claim 16, further comprising providing the first and second wire portions as coils.

18. The method of claim 17, further comprising providing the first and second wire portions comprising a superelastic material.

19. The method of claim 18, further comprising providing the first and second wire portions as being formed of Nitinol.

20. The method of claim 19, further comprising providing the first and second wire portions as extending through the tube, and providing the first and second wire portions as being unitarily formed as one piece.

21. The method of claim 20, further comprising providing the first wire portion extending substantially within a first plane and the second wire portion extending substantially within a second plane when the pressure equalization tube assembly is placed through the membrane in a working configuration.

22. The method of claim 21, further comprising providing the tube as having a circular cylindrical shape and being formed of a polymeric material.

23. A method of ventilating a tympanic membrane of an ear of a patient, the method comprising:
creating an incision within the tympanic membrane of the ear of the patient;
collapsing first and second coil portions of a pressure equalization tube assembly in a delivery tube;
inserting the delivery tube through the incision;
deploying the first coil portion from the delivery tube on an inner side of the tympanic membrane, the first coil portion expanding upon deployment and assuming a first coil expanded diameter;
retracting the delivery tube from the incision; and
deploying the second coil portion from the delivery tube on an outer side of the tympanic membrane, the second coil portion expanding upon deployment and assuming a second coil expanded diameter,
wherein the step of creating the incision includes creating the incision as being smaller than the each of the first and second coil expanded diameters.

24. The method of claim 23, further comprising providing the pressure equalization tube assembly having a tube disposed between the first and second coil portions, the tube having a tube diameter, the tube having an interior wall surface extending between a first end and a second end, the interior wall surface defining a lumen extending between the first and second ends, the first coil portion extending from the first end of the tube, and the second coil portion extending from the second end of the tube.

25. The method of claim 24, wherein the step of creating the incision includes creating the incision as being larger than the tube membrane and smaller than each of the first and second coil expanded diameters.

26. The method of claim 25, wherein the step of collapsing the first and second coil portions in the delivery tube includes providing the first coil portion with a first coil collapsed diameter and the second coil portion with a second coil collapsed diameter, wherein each of the first and second coil collapsed diameters are no larger than twice the tube diameter.

27. A method of ventilating a tympanic membrane of an ear of a patient, the method comprising:
creating an incision within the tympanic membrane of the ear of the patient;
inserting the delivery tube through the incision;
deploying a first coil portion of a pressure equalization tube assembly from the delivery tube on an inner side of the tympanic membrane, the first coil portion expanding upon deployment and assuming a first coil expanded diameter;
retracting the delivery tube from the incision; and
deploying a second coil portion of the pressure equalization tube assembly from the delivery tube on an outer side of the tympanic membrane, the second coil portion expanding upon deployment and assuming a second coil expanded diameter,
wherein the step of creating the incision includes creating the incision as being smaller than the each of the first and second coil expanded diameters.

28. The method of claim 27, further comprising providing the pressure equalization tube assembly having a tube disposed between the first and second coil portions, the tube having a tube diameter, the tube having an interior wall surface extending between a first end and a second end, the interior wall surface defining a lumen extending between the first and second ends, the first coil portion extending from the first end of the tube, and the second coil portion extending from the second end of the tube.

29. The method of claim 28, wherein the step of creating the incision includes creating the incision as being larger than the tube membrane and smaller than each of the first and second coil expanded diameters.

30. The method of claim 29, further comprising providing the first coil portion with a first coil collapsed diameter and the second coil portion with a second coil collapsed diameter prior to deploying the first and second coil portions from the delivery tube, wherein each of the first and second coil collapsed diameters are no larger than twice the tube diameter.
